(19) United States
(12) Patent Application Publication
(10) Pub. No.: US 2010/0204195 A1
(43) Pub. Date: Aug. 12, 2010

(54) Pharmaceutical Compositions and Process for Making Them

(75) Inventors: Amar Lulla, Mumbai (IN); Geena Malhotra, Mumbai (IN)

Correspondence Address: CONLEY ROSE, P.C., 5601 GRANITE PARKWAY, SUITE 750, PLANO, TX, 75024, US

(73) Assignee: CIPLA LIMITED, Mumbai (IN)

(21) Appl. No.: 12/670,717

(22) PCT Filed: Jul. 28, 2008
(86) PCT No.: PCT/GB08/02567

§ 371 Date: Apr. 7, 2010

(30) Foreign Application Priority Data
Jul. 27, 2007 (IN) 1446/MUM/2007
Jul. 31, 2007 (IN) 1450/MUM/2007
Dec. 12, 2007 (IN) 2432/MUM/2007

(51) Int. Cl. A61K 31/397; A61K 31/505; A61K 31/4418; A61K 31/47; A61K 31/44; A61K 31/404; A61K 31/40; A61K 31/366; A61K 31/216; A61K 31/192; A61P 9/00; A61P 3/00; A61P 3/10; A61P 9/10

(52) U.S. Cl. 514/210.02; 514/275; 514/277; 514/311; 514/356; 514/419; 514/423; 514/460; 514/533; 514/543; 514/571

(57) ABSTRACT

Amorphous HMG CoA reductase inhibitors, especially amorphous atorvastatin, are described. Also described are pharmaceutical combinations comprising amorphous HMG CoA reductase inhibitors in combination with cholesterol absorption inhibitors or fibrates. A method of manufacturing the compositions using a hot melt extrusion process are also described.

### CROSS-REFERENCE TO RELATED APPLICATIONS

This application is a filing under 35 U.S.C. 371 of International Application No. PCT/GB2008/002567 filed Jul. 28, 2008, entitled “Pharmaceutical Compositions and Process for Making Them,” claiming priority of Indian Patent Application Nos. 1446/MUM/2007 filed Jul. 27, 2007, 1450/MUM/2007 filed Jul. 31, 2007, and 2432/MUM/2007 filed Dec. 12, 2007, which applications are incorporated by reference herein in their entirety.

### FIELD OF THE INVENTION

The present invention relates to pharmaceutical compositions comprising one or more cholesterol reducing agents in an amorphous form. The invention also relates to processes for the preparation of the pharmaceutical compositions.

### BACKGROUND OF THE INVENTION

Cholesterol is a chemical that can both benefit and harm the body. On the good side, cholesterol plays important roles in the structure of cells and in the production of hormones. But too much cholesterol in the blood can lead to heart and blood vessel disease. One type, of cholesterol called high-density lipoprotein (HDL) cholesterol, or “good cholesterol,” actually lowers the risk of these problems but the other type, low-density lipoprotein (LDL) cholesterol, or “bad cholesterol,” is the type that threatens people's health. Hypercholesterolemia and hyperlipidemia, conditions of excessively high levels of blood cholesterol and lipids, are well recognized risk factors in the onset of atherosclerosis and coronary heart disease.

For several decades, it has been known that elevated blood cholesterol is a major risk factor for coronary heart disease (CHD), and many studies have shown that the risk of CHD events can be reduced by lipid-lowering therapy. Prior to 1987, the lipid-lowering armamentarium was limited essentially to a low saturated fat and cholesterol diet, the bile acid sequestrants (cholestyramine and colestipol), nicotinic acid (niacin), the fibrates and probucol. Unfortunately, all of these treatments have limited efficacy or tolerability, or both. Substantial reductions in LDL (low density lipoprotein) cholesterol accompanied by increases in HDL (high density lipoprotein) cholesterol could be achieved by the combination of a lipid-lowering diet and a bile acid sequestrant, with or without the addition of nicotinic acid. However, this therapy is not easy to administer or tolerate and was therefore often unsuccessful except in specialist lipid clinics. The fibrates produce a moderate reduction in LDL cholesterol accompanied by increased HDL cholesterol and a substantial reduction in triglycerides, and because they are well tolerated these drugs have been more widely used. Probucol produces only a small reduction in LDL cholesterol and also reduces HDL cholesterol, which, because of the strong inverse relationship between HDL cholesterol level and CHD risk, is generally considered undesirable. With the introduction of lovastatin, the first inhibitor of HMG-CoA reductase to become available for prescription in 1987, for the first time physicians were able to obtain large reductions in plasma cholesterol with very few adverse effects.

There are different types of cholesterol reducing agents that can be used.

One such type is HMG-CoA reductase inhibitors, often called “statins,” which lower the level of cholesterol in the blood by reducing the production of cholesterol by the liver. Statins block the enzyme hydroxy-methylglutaryl-coenzyme A reductase (HMG-CoA reductase) in the liver. Scientifically, statins are referred to as HMG-CoA reductase inhibitors. Statins are widely known for the treatment or prophylaxis of hyperlipidemia. Statins are the most effective cholesterol lowering agents available and in recent years have received increased attention for their benefits beyond helping patients with high cholesterol. Drugs in this group include: atorvastatin; cerivastatin; fluvastatin; lovastatin; pravastatin; simvastatin; and rosuvastatin and the like.

Atorvastatin calcium is [R-(R*,R*)]-2-(4-fluorophenyl)-(beta), [delta]-dihydroxy-5-(1-methylethyl)-3-phenyl-4-[(phenylamino)carbonyl]-1H-pyrrole-1-heptanoic acid, calcium salt (2:1) trihydrate. Atorvastatin calcium as a new chemical entity is described in U.S. Pat. No. 5,273,995.

Treatment with statins results in significant lowering of LDL cholesterol. This leads to significant reductions in cardiovascular morbidity and mortality. However, even with low levels of LDL cholesterol, patients with diabetes and metabolic syndrome continue to have a relatively high cardiovascular event rate, mainly due to the presence of atherogenic dyslipidemia, characterized by high triglycerides and small dense LDL and low HDL. Treatment with fibrates results in a substantial decrease in plasma triglycerides and is usually associated with a moderate decrease in LDL cholesterol and an increase in HDL cholesterol concentrations An important clinical challenge exists in reducing residual (coronary artery disease) (CAD) risk with optimal therapies without increasing adverse effects.

Another class of drugs, i.e. certain hydroxy-substituted azetidinones such as ezetimibe (described in U.S. Pat. No. 5,767,115 and Re. 37721), also known as cholesterol absorption inhibitors are known to be useful as hypocholesterolemic agents in the treatment and prevention of atherosclerosis. Cholesteryl esters are a major component of atherosclerotic lesions and the major storage form of cholesterol in arterial wall cells. Thus, inhibition of cholesteryl ester formation and reduction of serum cholesterol is likely to inhibit the progression of atherosclerotic lesion formation, decrease the accumulation of cholesteryl esters in the arterial wall, and block the intestinal absorption of dietary cholesterol.

“Fibrates” are a class of drugs that lower blood triglyceride levels by reducing the liver's production of VLDL (the triglyceride-carrying particle that circulates in the blood) and by speeding up the removal of triglycerides from the blood. Fibrates are also modestly effective in increasing blood HDL cholesterol levels; however, fibrates are not effective in lowering LDL. Fibrates are widely known for the treatment or prophylaxis of hyperlipidemia.

US 2005/0171207 relates to a pharmaceutical composition comprising a combination of R-Flurbiprofen and an HMG CoA reductase inhibitor and method of treating Alzheimer's disease.

WO2005/097191 relates to a pharmaceutical composition comprising optically pure (S)-amlodipine and a HMG CoA reductase inhibitor. The application also discloses a composition comprising optically pure (S)-amlodipine and a cholesterol absorption inhibitor.

WO2003/013608 discloses to a semisolid dosage formulation comprising fibrates and statins. However, manufacturing process involves melting the ingredients and filling in capsules, which is deleterious to some of the active ingredients. Also, the assessment of the quality of semi-solid lipid based formulations is quite difficult since the in vitro dissolution test is of little help. Indeed, the in vitro/in vivo correlation between dissolution and bioavailability is very poor for the kind of formulation given in the invention. Only pharmacokinetic studies on human subjects are reliable to assess the bioavailability of the drug.

U.S. Pat. No. 6,534,088 discloses an orally administered pharmaceutical composition comprising a statin and fenofibrate in the form of microparticles of solid fenofibrate that are stabilized by phospholipid as a surface active substance. The microparticles have been prepared by a process such as homogenization, microfluidization, hot melt microfluidization, and sonication so that the bioavailability of the drugs given in the dosage form improves. However, the formulation of the invention includes a range of excipients other than active ingredients. Further, the method of preparation of microparticles may lead to inactivation of some ingredients.

US2007/099891 discloses a pharmaceutical composition comprising a statin and/or ezetimibe and ACAT inhibitor (Acyl co-enzyme A cholesterol O acyl transferase inhibitor).

U.S. Pat. No. 7,229,982 relates to a pharmaceutical composition comprising ezetimibe, simvastatin, BHA and citric acid in specific concentration.

Environmental influences may degrade not only the active substance(s) in a pharmaceutical dosage form, undergoes degradation by environmental influences but also the excipient(s) in a pharmaceutical dosage form may be degraded. The degradation products of the latter may act as the reactive sites which trigger degradation reactions of the active substance in a pharmaceutical dosage form.

Among the environmental factors which have an impact on an active substance are, for example, temperature, humidity, light, (e.g., UV light) and gases, present in the environment such as, e.g., oxygen or carbon dioxide. An important factor is also the pH of the environment, that is, the presence of substances which have influence on the acidity or alkalinity of the environment (e.g., acids, alkalis, salts, metal oxides) and the reactivity of the ambient medium or active substance (free radicals, heavy metals), etc.

It is well known that for prevention or reduction degradation of active substances from getting degraded due to oxidation, various approaches are used, such as the use of antioxidants which in turn reduces the formation of peroxides (formation of which causes degradation); or addition of chelating agents to the formulation for mitigation of metallic impurities.

Thus, there is a need of a pharmaceutical composition which stands to minimize the risks of degradation of the active agents with incorporation of optimum excipients as pharmaceutical aids.

We have developed a pharmaceutical composition which can remain stable even in the absence of wherein the above additives, such as (i.e. antioxidants and, chelating agents) are not incorporated, and the composition still remains stable with respect to degradation of actives.

It is well known that active substances in an amorphous form have better solubility and dissolve more rapidly than in a crystalline form and thus have better bioavailability. The advantage of an amorphous active substance over a crystalline form is particularly evident in case of less soluble substances such as, for example, atorvastatin calcium, and it is manifested in better bioavailability of an active substance.

The use of a pharmaceutical formulation comprising amorphous active substance is advantageous over a pharmaceutical formulation comprising a crystalline substance because the amorphous substance dissolves faster and better which is an important factor for bioavailability of the active substance in the body. It is well known that the stability of an active substance depends on the polymorphous form in which it exists and that an amorphous form is less stable than a crystalline form indicating that an amorphous form compared to a crystalline form is even more susceptible to heat, light, moisture and low pH. All these factors are of key importance for the stability of a pharmaceutical formulation comprising an amorphous substance. Impurities generated at degradation of an active substance reduce the therapeutic effect of an active substance and additionally unnecessarily burden the body with unnecessary degradation products.

Atorvastatin calcium can exist in an amorphous form or in different crystalline forms which are disclosed in the patent applications WO 97/3958; WO 97/3959; WO 01/36384; WO 02/41834; WO 02/43732; WO 02/51804; and WO 02/57229. The processes for the preparation of amorphous atorvastatin calcium are described in the patent applications WO 97/3960; WO 00/71116; WO 01/28999; WO 01/42209; WO 02/57228; and WO 02/59087.

U.S. Pat. No. 7,151,183 relates to the preparation of amorphous atorvastatin calcium by dissolving the crystalline form in acetone, and then recovering the amorphous form from acetone.

U.S. Pat. No. 7,230,120 relates to preparation of atorvastatin calcium using methanol.

U.S. Pat. No. 6,528,660 relates to dissolving crystalline atorvastatin calcium in a non hydroxylic solvent, then adding a nonpolar hydrocarbon anti-solvent to precipitate out amorphous atorvastatin calcium.

US20070066835 relates to a process of for making the amorphous form by dissolving the salt in a mixture of water and water miscible organic solvent.

It is known from the patent and relevant other literature that atorvastatin calcium is an unstable substance which is susceptible to heat, moisture, light and low pH at which atorvastatin calcium is converted from the carboxylic acid form to the lactone form (U.S. Pat. No. 5,686,104). The problem of instability of atorvastatin calcium has been solved thus far by the addition of excipients to a pharmaceutical formulation with special emphasis to stabilization of atorvastatin calcium in the sense of conversion into the lactone form by the addition of a basifying or a buffering agent to the pharmaceutical composition (WO 00/35425; WO 94/16603). A procedure for stabilization of an active substance is known when in the final phase of synthesis an alkaline substance or a buffering solution is added to prepare an alkaline stabilized substance as described in the patent application WO 01/93860.

US20040077708 relates to a process for preparation of the stable pharmaceutical formulation comprising atorvastatin calcium in an amorphous form and pharmaceutically acceptable excipients which requires stringent condition of storing the pharmaceutical formulation in an inert atmosphere thereby achieving the stability which is superior and/or equal to the stability of the pharmaceutical formulation comprising the crystalline active substance.

To date, an appropriate and useful pharmaceutical compositions comprising atorvastatin calcium, Ezetimibe and fenofibrate have not been described.

Therefore, there is a need for preparing a stable pharmaceutical composition comprising amorphous active. Thus the present invention provides a pharmaceutical composition comprising an amorphous substance, the amorphous form being advantageous over a crystalline substance form by better bioavailability, and which is prepared according to the process which is simple and economically convenient.

### OBJECT OF THE INVENTION

An object of the invention is to provide new pharmaceutical formulations and methods of making them, to solve the problems in the prior art. The invention makes possible the manufacture of pharmaceutical compositions comprising one or more cholesterol reducing agent in an amorphous form, such as atorvastatin calcium, ezetemibe, fenofibrate and the like.

### SUMMARY OF THE INVENTION

The present invention relates to a pharmaceutical composition comprising one or more cholesterol reducing agents in an amorphous form, wherein at least one agent is converted in-situ from a non-amorphous form to an amorphous form during manufacture of the composition

The invention further relates to a composition comprising an extruded article containing one or more cholesterol reducing agent in an amorphous form in combination with a polymeric carrier, and optionally at least one pharmaceutically acceptable excipient.

The invention also relates to a method of making a pharmaceutical composition containing one or more amorphous cholesterol reducing agents, comprising blending the actives with a pharmaceutically acceptable polymer and optionally one or more pharmaceutically acceptable excipients, and hot melt extruding the blend to form an extrudate.

### BRIEF DESCRIPTION OF THE DRAWINGS

Reference is made to the accompanying drawing, in which:

FIG. 1 indicates an X-ray powder diffractogram (XRD) of crystalline atorvastatin calcium and X-ray powder diffractograms (XRD) of amorphous atorvastatin calcium.

### DETAILED DESCRIPTION OF THE INVENTION

In one aspect of the present invention, there is provided a pharmaceutical composition comprising an active substance which is an HMG CoA reductase inhibitor, preferably an amorphous HMG CoA reductase, wherein the active material is formed in-situ during the manufacturing of the composition.

In another aspect of the present invention, there is provided a pharmaceutical composition comprising one or more HMG CoA reductase inhibitors and one or more cholesterol absorption inhibitors and optional pharmaceutically acceptable excipients wherein either or both the active materials are preferably formed to an amorphous form in-situ during the manufacturing of the composition. The composition may be formulated for simultaneous, separate or sequential administration. The HMG CoA reductase inhibitor is preferably atorvastatin or a salt thereof, more particularly atorvastatin calcium. Most preferably, the cholesterol absorption inhibitor is ezetimibe or salt thereof. Most preferably, the HMG CoA reductase inhibitor is amorphous.

In yet another aspect of the present invention, there is a provided pharmaceutical composition comprising combination of one or more HMG CoA reductase inhibitors and one or more fibrates, wherein either or both the active materials are preferably formed to an amorphous form in-situ during the manufacturing of the composition. Optionally, the composition may further include one or more excipients. The composition may be formulated for simultaneous, separate or sequential administration. Most preferably, the HMG CoA reductase inhibitor is atorvastatin, or a salt thereof, especially atorvastatin calcium. Most preferably, the fibrate is fenofibrate or a salt thereof. Most preferably, the HMG CoA reductase inhibitor is amorphous.

In yet another aspect of the present invention, there is provided a method to manufacture the formulations according to the present invention.

The HMG CoA reductase inhibitor may be provided as the free material, or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. This applies to any of the materials used as the HMG CoA reductase inhibitor, including cerivastatin, atorvastatin, lovastatin, simvastatin, rosuvastatin, pravastatin, mevastatin, fluvastatin, rivastatin, pitavastatin.

The cholesterol absorption inhibitor may be provided as the free material, or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. This applies to any of the materials used as the cholesterol absorption inhibitor, including ezetimibe.

The fibrate may be provided as the free material, or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. This applies to any of the materials used as the cholesterol absorption inhibitor, including ezetimibe.

Broadly, the invention relates to pharmaceutical compositions containing a cholesterol reducing agent, and to methods of manufacturing the composition. The cholesterol reducing agent is preferably one or more HMG CoA reductase inhibitor; one or more cholesterol absorption inhibitor; one or more fibrate; or a combination of two or more thereof. In particular, the invention envisages the provision of a pharmaceutical combination comprising one or more HMG CoA reductase inhibitors in combination with one or more cholesterol absorption inhibitors; and the invention also envisages the provision of a pharmaceutical combination comprising one or more HMG CoA reductase inhibitors in combination with one or more fibrates.

The different active materials in the pharmaceutical composition may be provided in a single unitary dosage form, or may be provided in separate dosage forms for separate or sequential administration.

Each of the cholesterol reducing agents may be provided in an amorphous form. It is possible for one or more of the cholesterol reducing agents to be provided in an amorphous form, with other cholesterol reducing agents.

It is a feature of the invention that the amorphous form of the cholesterol reducing agents is formed during formation of the pharmaceutical composition. Most preferably, the amorphous form is formed in-situ during formation of the pharmaceutical composition.

In another aspect, the pharmaceutical composition may comprise an extrudate formed of at least one cholesterol reducing agent in combination with a pharmaceutically acceptable polymer and optional pharmaceutically acceptable excipients.

In another aspect, the invention relates to a pharmaceutical precursor composition comprising an extrudate formed of at least one cholesterol reducing agent in combination with a pharmaceutically acceptable polymer and optional pharmaceutically acceptable excipients. The extrudate may be subsequently processed to produce a pharmaceutical composition. This subsequent processing may involve the addition of one or more cholesterol reducing actives (especially HMG CoA reductase inhibitors, cholesterol absorption inhibitor and/or fibrates), which may each be in amorphous or non-amorphous form. The subsequent processing may also involve the addition of one or more pharmaceutically acceptable excipients (further to any excipients already present in the extrudate).

Thus, the invention encompasses an extruded article containing at least one cholesterol reducing agent (especially a HMG CoA reductase inhibitor, a cholesterol absorption inhibitor and/or a fibrate). This extrudate typically further comprises a pharmaceutically acceptable polymer in combination with the actives, which are suitably homogeneously dispersed throughout the polymer. The extrudate may also include one or more pharmaceutically acceptable excipients. At least one of the actives, and possibly all of the actives, are preferably present in the extrudate in amorphous form. The amorphous form was preferably formed during the manufacture of the extrudate (i.e., the starting material actives were in form different from the amorphous form).

The pharmaceutical composition according to the invention may be provided in a suitable form, including, but not limited to: tablet, capsule, pellet, sprinkles, powder; granules; sachet; or in the form of a liquid oral dosage solution or suspension.

In accordance with one aspect of the invention, there is provided a pharmaceutical composition comprising one or more cholesterol reducing agent in an amorphous form, wherein at least one active is converted in-situ to an amorphous form during the manufacture of the composition.

Preferably, the pharmaceutical composition further comprises a polymer.

In accordance with another aspect of the invention there is provided a composition comprising an extruded article containing one or more cholesterol reducing agent in an amorphous form in combination with a polymeric carrier, and optionally at least one pharmaceutically acceptable excipient. The extruded article is advantageously formed by a hot melt extrusion process, as described below.

Preferably, at least one amorphous agent is formed in situ during the manufacture of the composition.

In an embodiment, the ratio of agent to polymer is from 1:1 to 1:6 on a weight basis, preferably 1:2 to 1:5 on a weight basis.

In an embodiment, the composition comprises 2 to 80 wt % polymer, preferably 20-80% by weight of polymer.

The extruded article may be a pharmaceutical composition, per se. Alternatively, a pharmaceutical composition according to the invention may be obtained from the extruded article by appropriate processing and optional mixing with at least one pharmaceutically acceptable excipient.

The pharmaceutical compositions according to the invention may, for example, be formed from an extrudate containing both actives; or from two separate extrudates, each containing one active; or from an extrudate containing one or more actives, which is subsequently processed with one or more other actives not provided as extrudates using hot melt technique. The active materials may be homogeneously mixed in the dosage form. Alternatively, the active materials along with one or more excipients may be provided in separate individual layers in the tablet, such as bilayer form for two actives, trilayer form for three actives, and so on.

In the compositions described above, the cholesterol reducing active preferably comprises at least one HMG-CoA inhibitor, at least one cholesterol absorption inhibitor and/or at least one fibrate.

In an especially preferred embodiment, the cholesterol reducing agent comprises at least one HMG CoA reductase inhibitor, at least one cholesterol absorption inhibitor and/or at least one fibrate. The composition may include just one active material selected from those mentioned above. Alternatively, there may be more than one of the same type of active. Alternatively, there may be different types of active, or more than one of different types of active.

The fibrate is preferably selected from benzafibrate, gemfibrozil, fenofibrate, simfibrate, ronifibrate, ciprofibrate, etofibrate, clofibrate, clinofibrate, most preferably, the fibrate is fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. As noted above, the fibrate may be in amorphous form, and it is preferably converted to an amorphous form in situ during manufacture of the composition.

In another especially preferred embodiment, the cholesterol reducing agent comprises at least one HMG CoA reductase inhibitor and/or at least one cholesterol absorption inhibitor.

The cholesterol absorption inhibitor is preferably ezetimibe or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. As noted above, the cholesterol absorption inhibitor may be in amorphous form, and it is preferably converted to an amorphous form in situ during manufacture of the composition.

In the above embodiments, the HMG CoA reductase inhibitor is selected from cerivastatin, atorvastatin, lovastatin, simvastatin, rosuvastatin, pravastatin, mevastatin, fluvastatin, rivastatin, pitavastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. The preferred HMG CoA reductase inhibitor is atorvastatin or a salt thereof, most preferably atorvastatin calcium. As noted above, the HMG CoA reductase inhibitor may be in amorphous form, and it is preferably converted to an amorphous form in situ during manufacture of the composition.

In one particularly preferred embodiment of the invention, the cholesterol reducing agent comprises atorvastatin calcium and ezetimibe in combination. In another particularly preferred embodiment of the invention, the cholesterol reducing active comprises atorvastatin calcium and fenofibrate. In either embodiment, the active material may be formulated for simultaneous, sequential or separate administration. They are preferably formulated in a single dosage form. They may be formed from an extrudate containing both actives; or form two separate extrudates, each containing one active; or from an extrudate containing one active, which is subsequently processed with the other active not provided as an extrudate. The active materials may be homogeneously mixed in the dosage form. Alternatively, the active materials may be provided in separate individual layers in the tablet, such as a bilayer tablet form. In either case, at least one of the actives (and possibly both) is in amorphous form, and the amorphous form is preferably formed, in-situ during manufacture.

The compositions according the invention are preferably obtainable by melt extruding at least one cholesterol reducing agent along with at least one pharmaceutically acceptable polymer, and optionally one or more other pharmaceutically acceptable excipients, to form an extrudate, optionally treating the extrudates to suitable solid forms and further, optionally admixing the extrudate with one or more further pharmaceutically acceptable excipients.

The extrudate, and optional excipient or excipients, may be further treated to be filled into a suitable pharmaceutical receptacle, such as a sachet or capsule, pellets, sprinkles, oral suspensions.

The extrudate, and optional excipient or excipients, may be processed, for example by compression, to form a tablet.

According to another aspect of the invention, there is provided a method of making a pharmaceutical composition containing one or more cholesterol reducing actives, of which at least one and possible all are preferably in an amorphous form, comprising blending the actives with a pharmaceutically acceptable polymer, and hot melt extruding the blend to form an extrudate.

In a preferred embodiment, at least one cholesterol reducing agent, and possibly all of them, is blended with the polymer optionally with one or more excipients, and the amorphous form of said active is formed in-situ during the hot melt extrusion.

In the method, the cholesterol reducing active comprises a HMG-CoA reductase inhibitor, a cholesterol absorption inhibitor and/or a fibrate, as described above in respect of the compositions.

In a preferred embodiment, the extrudate is processed and optionally mixed it with one or more additives to form the pharmaceutical composition.

In an embodiment, the method further comprises the steps of cutting the extrudate to a desired shape to form a pharmaceutical composition.

In an embodiment, the method further comprises treating the extrudate to form a granulate. The granulate may be filled into a capsule or sachet, or process into a tablet, e.g., by compression.

The compositions according to the invention may be used as a medicament, in particular to treat various disorders due to an increase in cholesterol. In particular, the compositions may be used to treat dyslipidemia, hyperlipidemia, hypercholesterolemia, atherosclerosis, arteriosclerosis, cardiovascular disease, coronary artery disease, coronary heart disease, vascular disorder and/or related disorders. The treatment may be achieved by administering a therapeutically effective amount of the pharmaceutical composition of the present invention to a mammal in need thereof in a suitable therapeutic regimen.

As described above, amorphous forms have better solubility and dissolve more rapidly than in a crystalline form.

We have surprisingly found that when crystalline actives like HMG-CoA reductase inhibitors, cholesterol absorption inhibitors, fibrates are formulated by hot melt extrusion, the resultant product is a formulation containing the amorphous active which remains stable.

Thus, the present invention provides an economical and easy way to formulate a stable formulation.

The term HMG CoA reductase inhibitors cholesterol absorption inhibitors, fibrates are mentioned in the description as well as the claims in a broad sense to include not only HMG CoA reductase inhibitors cholesterol absorption inhibitors, fibrates per se, but also its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

According to the present invention, various HMG CoA reductase inhibitors can be used. These include atorvastatin, lovastatin, simvastatin, rosuvastatin, pravastatin, mevastatin, fluvastatin, rivastatin, pitavastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. Most preferably, the statins are amorphous.

Thus, to summarise, the cholesterol reducing agent may comprise at least one HMG-CoA inhibitor, at least one cholesterol absorption inhibitor and/or at least one fibrate. One or more of these agents may be in an amorphous form, and the amorphous form may be formed in-situ during manufacturing. Preferably, the cholesterol reducing agent comprises (i) at least one HMG-CoA inhibitor and at least one cholesterol absorption inhibitor; or (ii) at least one HMG-CoA inhibitor and at least one fibrate. The fibrate is preferably selected from benzafibrate, gemfibrozil, fenofibrate, simfibrate, ronifibrate, ciprofibrate, etofibrate, clofibrate, clinofibrate, with fenofibrate being particularly preferred. The cholesterol absorption inhibitor is preferably ezetimibe. The HMG CoA reductase inhibitor is preferably selected from cerivastatin, atorvastatin, lovastatin, simvastatin, rosuvastatin, pravastatin, mevastatin, fluvastatin, rivastatin, pitavastatin, with atorvastatin, especially atorvastatin calcium, being particular preferred. The combination of atorvastatin, especially atorvastatin calcium, with fenofibrate is particularly preferred. The combination of atorvastatin, especially atorvastatin calcium, with ezitimbe is also particularly preferred.

The invention encompasses using a pharmaceutical acceptable salt of any of these agents.

The invention encompasses using a pharmaceutically acceptable solvate of any of these agents.

The invention encompasses using a pharmaceutically acceptable enantiomer of any of these agents.

The invention encompasses using a pharmaceutically acceptable derivative of any of these agents.

The invention encompasses using a pharmaceutically acceptable polymorph of any of these agents.

The invention encompasses using a pharmaceutically acceptable prodrug of any of these agents.

Further, it has been known that a major problem with amorphous HMG-CoA reductase inhibitors is their stability when formulated per se or when combined with other actives.

From the prior art, it is understood that to prevent degradation of the actives due to oxidation, the use of additives like chelating agents or antioxidants are incorporated. These are not needed to maintain stability in the compositions according to the present invention. Thus, it is preferred that the composition according to the invention does not contain a chelating agent. It is preferred that, if the composition according to the invention does contain an antioxidant or chelating agent, the amount is not more than 0.1 to 2% by weight of the composition.

We have found that a stable composition is formed when such actives are formulated by hot melt extrusions.

Thus, one or more cholesterol reducing agents like HMG-CoA reductase inhibitors, cholesterol absorption inhibitors, fibrates can be combined together.

Thus, in one aspect of the present invention there is provided a pharmaceutical composition comprising one or more HMG CoA reductase inhibitors and one or more cholesterol absorption inhibitors and optional pharmaceutically acceptable excipients wherein either or both the active materials are formed to an amorphous form in-situ during the manufacturing of the composition. The composition may be formulated for simultaneous, separate or sequential administration.

The HMG CoA reductase inhibitors according to the present invention may be selected from atorvastatin, lovastatin, simvastatin, rosuvastatin, pravastatin, mevastatin, fluvastatin, rivastatin, pitavastatin.

The pharmaceutical composition according to the present invention comprises at least one cholesterol absorption inhibitor, such as ezetimibe or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

The quantity of the cholesterol absorption inhibitor to be used in the formulation preferably ranges from 1% to 15% by weight of the composition.

The quantity of the HMG CoA reductase inhibitor to be used in the formulation is preferably from 1% to 15% by weight of the composition.

The preferred formulation according to the invention comprises atorvastatin, or a salt thereof, most preferably atorvastatin calcium, in combination with ezetimibe. And most preferably the atorvastatin, or atorvastatin calcium is amorphous and has preferably been produced in accordance with the methods described herein.

In one embodiment, the cholesterol absorption inhibitor (e.g. ezetimibe) may be manufactured using a hot melt extrusion process as described above. This may be done in combination with the HMG CoA reductase inhibitor, or separately.

The combination therapy of one or more HMG CoA reductase inhibitors, and one or more fibrates, preferably with one or more pharmaceutically acceptable excipients, improves the lipid profiles i.e decrease in triglycerides, moderate decrease in LDL cholesterol, increase in HDL cholesterol that appear to be frequently necessary for high risk CAD patients.

Thus, in another aspect of the present invention, there is a provided pharmaceutical composition comprising combination of one or more HMG CoA reductase inhibitors and one or more fibrates, wherein either or both the active materials are formed to an amorphous form in-situ during the manufacturing of the composition. Optionally, the composition may further include one or more excipients. The composition may be formulated for simultaneous, separate or sequential administration.

The HMG CoA reductase inhibitors according to the present invention can be selected from atorvastatin, lovastatin, simvastatin, rosuvastatin, pravastatin, mevastatin, fluvastatin, rivastatin, pitavastatin.

The fibrate, according to the present invention, may be selected from, but is not limited to benzafibrate, gemfibrozil, fenofibrate, simfibrate, ronifibrate, ciprofibrate, etofibrate, clofibride and the possible pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

According to a preferred embodiment, the pharmaceutical composition comprises fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

According to present invention, the composition comprising one or more statins preferably amorphous atorvastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof; and fibrates preferably fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with one or more pharmaceutically acceptable excipients wherein either or both of the active(s) are converted insitu to amorphous form during processing.

In another aspect of the present invention, the present invention relates to a method of making a pharmaceutical composition containing one or more amorphous cholesterol reducing actives, comprising blending the actives with a pharmaceutically acceptable polymer with one or more pharmaceutically acceptable excipients, and hot melt extruding the blend to form an extrudate.

During the hot melt extrusion process, the polymers that can be used according to the present invention, include water soluble and water insoluble polymers. Examples of suitable polymers include homopolymers and copolymers of N-vinyl pyrrolidone, e.g. polyvinylpyrrolidone (PVP); copolymers of N-vinyl pyrrolidone and vinyl acetate or vinyl propionate; cellulose esters and cellulose ethers, in particular methylcellulose and ethylcellulose; hydroxyalkylcelluloses, in particular hydroxypropylcellulose, hydroxyalkylalkylcelluloses, in particular hydroxypropylmethylcellulose; cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides, such as polyethylene oxide and polypropylene oxide and copolymers of ethylene oxide and propylene oxide; polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxylalkyl acrylates), poly(hydroxyalkyl methacrylates); polyacrylamides; vinyl acetate polymers such as copolymers of vinyl acetate and crotonic acid, and partially hydrolyzed polyvinyl acetate (also referred to as partially saponified (“polyvinyl alcohol”) polyvinyl alcohol.

The water soluble polymers that can be used, according to the present invention, comprises of homopolymers and co-polymers of N-vinyl lactams, especially homopolymers and co-polymers of N-vinyl pyrrolidone e.g. polyvinylpyrrolidone (PVP), co-polymers of PVP and vinyl acetate, co-polymers of N-vinyl pyrrolidone and vinyl acetate or vinyl propionate, esters and cellulose ethers, high molecular polyalkylene oxides such as polyethylene oxide and polypropylene oxide and co-polymers of ethylene oxide and propylene oxide.

The water insoluble polymer that can be used, according to the present invention, comprises of acrylic copolymers e.g. Eudragit E100 or Eudragit EPO; Eudragit L30D-55, Eudragit FS30D, Eudragit RL30D, Eudragit RS30D, Eudragit NE30D, Acryl-Eze (Colorcon Co.); polyvinylacetate, for example, Kollicoat SR 30D (BASF Co.); cellulose derivatives such as ethylcellulose, cellulose acetate e.g. Surelease (Colorcon Co.), Aquacoat ECD and Aquacoat CPD (FMC Co.).

Preferably, homopolymers or copolymers of N-vinyl pyrrolidone, hydroxyalkylcelluloses, polyacrylates are used.

Suitably, homopolymers or copolymers of N-vinyl pyrrolidone include a copolymer of N-vinyl pyrrolidone & vinyl acetate. A preferred polymer is a copolymer N-vinyl pyrrolidone wherein about 40% by weight of the copolymer is vinyl acetate.

Suitably, polyacrylates include cationic copolymers based on dimethylaminoethyl methacrylate and neutral methacrylic esters. More preferably, the polymer can be Eudragit E100.

Suitably, the hydroxyalkycellulose is hydroxypropylcellose.

The polymers can be present in a concentration of 2 to 80% by weight of the composition, preferably 20 to 80% by weight of the composition.

A single polymer may be used or a combination of one or more polymers may be used.

Different ratios of the drug: polymer may be used. Suitably, the ratio may be of the range of 1:1 to 1:6, preferably 1:2 to 1:5.

In addition to the drug and polymer, the blend may further comprise additional excipients like plasticizers, disintegrants, flow regulators, lubricants, fillers, stabilizers such as antioxidants, light stabilizers, radical scavengers, stabilizers against microbial attack. The composition may contain 0.5% to 10% alkalinizing agent by weight of the composition.

Plasticizers can be incorporated depending on the polymer and the process requirement. These, advantageously, when used in the hot melt extrusion process decrease the glass transition temperature of the polymer. Plasticizers also help in reducing the viscosity of the polymer melt and thereby allow for lower processing temperature and extruder torque during hot melt extrusion. Examples of suitable plasticizers which can be used in the present invention, include, but are not limited to, polysorbates such as sorbitan monolaurate (Span 20), sorbitan monopalmitate, sorbitan monostearate, sorbitan monoisostearate; citrate ester type plasticizers like triethyl citrate, citrate phthalate; propylene glycol; glycerin; low molecular weight polyethylene glycol; triacetin; dibutyl sebacate, tributyl sebacate; dibutyltartrate, dibutyl phthalate. The plasticizer is preferably present in an amount ranging from 0% to 10% to the weight of polymer.

Suitable flow regulators are selected from highly dispersed silica (Aerosil), and animal or vegetable fats or waxes.

Various other additives may be used, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack.

The present invention also provides a process to manufacture a composition according to the present invention. The process involves forming a powder blend, transferring the blend through a heated barrel of the extruder, whereby the powder blend melts and molten solution product is collected on a conveyor whereby it is allowed to cool and form an extrudate. Alternatively, the extrudate is cut into pieces after solidification and can be further processed into suitable dosage forms. More preferably the extrudates thus finally obtained from the above process are then milled and ground to granules or other solid forms by the means known to a person skilled in the art.

The extrudates so obtained may then be admixed with one or more other suitable pharmaceutically acceptable excipients.

Suitable bulking agents/diluents may include one or more of, but not limited to, dibasic calcium phosphate, calcium sulfate, microcrystalline cellulose, cellulose powdered, dextrates, dextrins, maltodextrin, dextrose excipients, croscarmellose sodium, isomalt, PVA, saccharides, including monosaccharides, disaccharides, polysaccharides and sugar alcohols such as arabinose, maltose, fructose, lactitol, lactose, mannitol, sorbitol, starch, starch pregelatinized, sucrose, sugar compressible, sugar confectioners and mixtures thereof. The diluent may be present in a quantity from 30% to 85% by weight of the composition.

Suitable binders may include one or more of, but not limited, to methyl cellulose, hydroxypropyl cellulose, hydroxypropyl methylcellulose, polyvinylpyrrolidone, gelatin, gum arabic, polyvinyl alcohol, pullulan, starch, pregelatinized starch, agar, tragacanth, sodium alginate, propylene glycol, alginate and other cellulose derivatives and equivalents thereof. Preferably the binder is present in a quantity from 1% to 15% by weight of composition.

Suitable disintegrants may include one or more of, but not limited, to starches, clays, celluloses, algins, gums or crosslinked polymers one or more of low substituted hydroxypropyl cellulose, carboxymethyl cellulose, calcium carboxymethyl cellulose, sodium carboxymethyl cellulose, sodium starch glycollate, crospovidone, croscarmellose sodium, starch, crystalline cellulose, hydroxypropyl starch, and partially pregelatinized starch. The disintegrant may be present in a quantity ranging from 1% to 10% by weight of the composition.

Suitable lubricants/glidants may include one or more of, but not limited to, stearic acid, magnesium stearate, calcium stearate, talc, hydrogenated caster oil, sucrose esters of fatty acid, PEG microcrystalline wax, colloidal silicon dioxide and equivalents thereof. Optionally, suitable coloring agents may be added. The formulation may incorporate one or more of the above lubricants or glidants. The glidant and lubricant may each be present in an amount from 0.5% to 5% by weight of the composition.

The composition may further comprise alkalinizing agents including, but not limited to, calcium carbonate, calcium phosphate, magnesium carbonate, magnesium oxide, potassium carbonate, potassium hydrogen carbonate, sodium carbonate, sodium hydrogen carbonate and equivalents thereof. The formulation may incorporate one or more of the above alkalinizing agents.

The different active materials in the pharmaceutical composition may provided in a single unitary dosage form, or may provided in separate dosage forms for separate or sequential administration

The pharmaceutical composition according to the invention may be provided in a suitable form, including, but not limited to: tablet, capsule, pellet, sprinkles, powder; granules; sachet; or in the form of a liquid oral dosage solution or suspension.

The pharmaceutical compositions according to the invention may, for example, be formed from an extrudate containing both actives; or from two separate extrudates, each containing one active; or from an extrudate containing one or more actives, which is subsequently processed with one or more other actives not provided as extrudates. The active materials may be homogeneously mixed in the dosage form. Alternatively, the active materials may be provided in separate individual layers in the tablet, such as bilayer form for two actives, trilayer form for three actives, and so on.

Further, the extrudates and the pharmaceutically acceptable excipients can be processed by techniques known to a person skilled in the art, such as direct compression, wet granulation, fluidized bed granulation, extrusion, solvent evaporation and are not intended to limit the scope of the invention to form the desired dosage form.

The present invention further provides for a method of treatment of various disorders due to increased cholesterol such as dyslipidemia, hyperlipidemia, hypercholesterolemia, atherosclerosis, arteriosclerosis, cardiovascular disease, coronary artery disease, coronary heart disease, vascular disorder and related disorders by administering a therapeutically effective amount of the pharmaceutical composition of the present invention to a mammal in need thereof in a suitable therapeutic regimen.

In general terms, the process of hot melt extrusion is carried out in the conventional extruders as known to a person skilled in the art.

The melt-extrusion process comprises the steps of preparing a homogeneous melt of one or more drugs, the polymer and the excipients, and cooling the melt until it solidifies. “Melting” means a transition into a liquid or rubbery state in which it is possible for one component to get embedded homogeneously in the other.

Typically, one component will melt and the other components will dissolve in the melt thus forming a solution. Melting usually involves heating above the softening point of the polymer. The preparation of the melt can take place in a variety of ways. The mixing of the components can take place before, during or after the formation of the melt. For example, the components can be mixed first and then melt extruded or be simultaneously mixed and melt extruded. Usually, the melt is homogenized in order to disperse the active ingredients efficiently. Also, it may be convenient first to melt the polymer and then to mix in and homogenize the active ingredients.

Usually, the melt temperature is in the range of about 70° C. to about 200° C., preferably from about 80° C. to about 180° C.

Suitable extruders include single screw extruders, intermeshing screw extruders or else multiscrew extruders, preferably twin screw extruders, which can be co-rotating or counter-rotating and, optionally, be equipped with kneading disks. It will be appreciated that the working temperatures will also be determined by the kind of extruder or the kind of configuration within the extruder that is used.

The extrudates can be in the form of beads, granulates, tube, strand or cylinder and this can be further processed into any desired shape.

The term “extrudates” as used herein refers to solid product solutions, solid dispersions and glass solutions of one or more drugs with one or more polymers and optionally pharmaceutically acceptable excipients.

According to a preferred embodiment, a powder blend of the one or more active drug(s) and polymers and optionally pharmaceutical excipients are transferred by a rotating screw of a single screw extruder through the heated barrel of an extruder whereby the powder blend melts and molten solution product is collected on a conveyor where it is allowed to cool to form an extrudate. Shaping of the extrudate can conveniently be carried out by a calendar with two counter-rotating rollers with mutually matching depressions on their surface.

A broad range of extrudated forms can be attained by using rollers with different forms of depressions. Alternatively, the extrudate is cut into pieces after solidification and can be further processed into suitable dosage forms. More preferably, the extrudates thus finally obtained from the above process are then milled and ground to granules by the means known to a person skilled in the art.

Further, hot melt extrusion is a fast, continuous, single pot manufacturing process without requirement of further drying or discontinuous process steps; it provides short thermal exposure of active which allows processing of heat sensitive actives; process temperatures can be reduced by addition of plasticizers; comparatively lower investment for equipment as against other processes. The entire process is anhydrous and the intense mixing and agitation of the powder blend that occur during processing contribute to a very homogenous extrudate(s).

In one aspect, the preferred embodiment in accordance with the present invention may comprise one or more statins and one or more fibrates or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof alongwith one or more water soluble polymers which are melt extruded by the process as described herein, where a powder blend of one or more statins, preferably, amorphous atorvastatin and one or more fibrates, preferably fenofibrate or their pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof along with one or more polymers and other excipients which may comprise suitable plasticizers and/or bulking agents. These are processed to form a powder blend which is transferred through the heated barrel of the extruder, whereby the powder blend melts and molten solution product is collected on a conveyor whereby it is allowed to cool and form an extrudate.

According to preferred embodiment, the formulation of the invention comprises blending of amorphous atorvastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more polymers and other excipients which may comprise suitable bulking agents and plasticizers. These are processed to form a powder blend which is transferred through the heated barrel of the extruder, whereby the powder blend melts and molten solution product is collected on a conveyor whereby it is allowed to cool and form an extrudate.

Alternatively, the extrudate is cut into pieces after solidification and can be further processed into suitable dosage forms. More preferably, the extrudates thus finally obtained from the above process are then milled and ground to granules by the means known to a person skilled in the art.

The extrudates thus obtained as described above, can be compressed as such to form tablets or incorporated as granules into various pharmaceuticals compositions that include, but are not limited to, tablets, capsules, pellets sprinkles, oral suspensions.

In another aspect, the preferred embodiment in accordance with the present invention may comprise one or statins and a combination of one or more water insoluble polymer and one or more water soluble polymer which are melt extruded by the process as described herein, where a powder blend of one or more statins or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof & fibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and a combination of water soluble polymer(s) & water insoluble polymer(s) and other excipients which may comprise suitable bulking agents and plasticizer.

According to preferred embodiment, the formulation of the invention comprises blending of amorphous atorvastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and a combination of one or more water insoluble polymer or combination of water soluble and water insoluble polymer and one or more pharmaceutically acceptable excipients which may comprise suitable bulking agents and plasticizers.

These are processed to form a powder blend which is transferred through the heated barrel of the extruder, whereby the powder blend melts and molten solution product is collected on a conveyor whereby it is allowed to cool and form an extrudate.

Alternatively, the extrudate is cut into pieces after solidification and can be further processed into suitable dosage forms. More preferably, the extrudates thus finally obtained from the above process are then milled and ground to granules or other solid forms by the means known to a person skilled in the art.

According to another embodiment, the present invention may further involve one or more manufacturing process to obtain a single unitary dosage form i.e. wherein the or each drug is processed by the techniques as discussed above and finally compacted to yield a single dosage form. Preferably, statin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs in combination with one or more excipients & fibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs in combination with one or more excipients may be processed with the techniques as discussed above separately and may be combined to form single unitary dosage form. Preferably, the statin blend is mixed with fibrate blend and may be compressed into a single-layered tablet. Alternatively, the statin blend may be compacted and compressed into a tablet and fibrate blend may be compacted and compressed into tablet and finally each individual layer may be compressed into a bilayer tablet. According to one aspect, the tablet may be seal coated. According to another aspect, the tablet may be seal coated and finally film coated. The formulation can be coated with Ready colour mix systems (such as Opadry colour mix systems).

Accordingly, the formulation of the present invention comprises blending of amorphous atorvastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with one or more pharmaceutically acceptable excipients, and fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with one or more pharmaceutically acceptable excipients. Further, the amorphous atorvastatin blend is mixed with fenofibrate blend and may be compacted and compressed into a tablet and finally each individual layer may be compressed into a bilayer tablet. According to one aspect, the tablet may be seal coated. According to another aspect, the tablet may be seal coated and finally film coated.

According to second embodiment, the present invention may be formulated wherein the or each drug, preferably, statin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients may be processed through wet granulation, direct compression and the like as mentioned above and fibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with one or more pharmaceutically acceptable excipients may be processed through melt granulation, melt extrusion and the like as mentioned above.

Accordingly, the formulation of present invention comprises amorphous atorvastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with one or more pharmaceutically acceptable excipients may be processed through wet granulation, direct compression and the like as mentioned above and fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with one or more pharmaceutically acceptable excipients may be processed through melt granulation, melt extrusion and the like as mentioned above.

Preferably, statin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs are mixed with intragranular excipients which includes, but not limited to, diluents, disintegrants and granulated with water or other aqueous solvents, sieved, sifted and lubricated and dried. Alternatively, the dried granules may be compressed into tablets.

More preferably, amorphous atorvastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs are mixed with intragranular excipients which includes, but not limited to, diluents, disintegrants and granulated with water or other aqueous solvents, sieved, sifted and lubricated and dried. Alternatively, the dried granules may be compressed into tablets.

Preferably, fibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs and one or more excipients which includes, but are not limited to, polymers (i.e. either water soluble or water insoluble or mixture thereof), one or more plasticizer, one or more disintegrants, one or more lubricants and glidants are extruded through hot melt extrusion technique wherein extrudates are obtained which can be molded into desired shapes that can be filled in sachets or can be granulated. Alternatively, the granules may be compressed into tablets.

More preferably, fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs and one or more excipients which includes, but are not limited to, polymers (i.e. either water soluble or water insoluble or mixture thereof), one or more plasticizer, one or more disintegrants, one or more lubricants and glidants are extruded through hot melt extrusion technique wherein extrudates are obtained which can be molded into desired shapes that can be filled in sachets or can be granulated. Alternatively, the granules may be compressed into tablets.

According to a preferred embodiment, the granules (comprising the individual actives) as obtained above may be further mixed, sieved, sifted and compressed into a single tablet or may be filled into capsules or sachets or the granules may be administered directly. Alternatively, the tablet may be seal coated and finally film coated.

Alternatively, the or each granules (comprising the individual actives) as obtained above may be individually compressed into two tablets and finally compacted and compressed into a bilayer tablet. Alternatively, the tablet may be seal coated and finally film coated.

### EXAMPLES

The following examples are for the purpose of illustration of the invention only and are not intended in any way to limit the scope of the invention.

### Examples 1-6

Exemplary compositions of the present invention for melt extrusion of atorvastatin calcium with one or more polymer (s) are shown below in table 1.

TABLE 1

| Sr. | | Example (mg) | | | | | |
|---|---|---|---|---|---|---|---|
| No. | Ingredients | 1 | 2 | 3 | 4 | 5 | 6 |
| 1 | Atorvastatin Ca | 80 | 80 | 80 | 80 | 80 | 80 |
| 2 | Hydroxypropyl | 320 | — | — | 80 | — | 160 |
| | cellulose | | | | | | |
| 3 | Copolymer of N-vinyl | — | 320 | — | 80 | 160 | — |
| | pyrrolidone & vinyl | | | | | | |
| | acetate (Kollidon | | | | | | |
| | VA 64 ®) | | | | | | |
| 4 | Dimethyl aminoethyl | — | — | 320 | — | 160 | 80 |
| | methacrylate ester | | | | | | |
| | (Eudragit E100 ®) | | | | | | |

The drug and polymer(s) were passed individually through 20 mesh. The drug(s) and polymer(s) were mixed and again passed through 20 mesh. The mixture was hot melt extruded at a temperature ranging from 60-160° C. Most preferably, at a temperature between 90-120 C. Optionally suitable plasticizers and surfactants were added in the hot melt extrusion process.

### Example 7

A composition according to the invention comprised the following components:

| Sr. | | Qty/Unit |
|---|---|---|
| No. | Ingredients | (mg) |
| 1 | Atorvastatin Calcium | 82.87 |
| 2 | PVP VA 64 | 320.00 |
| 3 | Span 20 | 32.00 |
| 4 | Calcium carbonate | 30.00 |
| 5 | LHPC | 80.00 |
| 6 | Crosscarmellose sodium | 50.00 |
| 7 | Talc | 16.00 |
| 8 | Pearlitol DC 400 (Mannitol) | 369.13 |
| 9 | Calcium Stearate | 20.00 |
| | Total | 1000.00 |

Atorvastatin Calcium, PVP VA-64 and Span 20 were hot melt extruded. The extrudates were sized and mixed with calcium carbonate, crosscarmellose sodium and LHPC. This was then diluted with Perlitol DC 400 and lubricated with talc and calcium stearate.

### Example 8

It was observed that when Crystalline Atorvastatin was processed using hot-melt extrusion, there was a conversion of the crystalline form to amorphous form and the final formulation that was obtained contained the amorphous Atorvastatin.

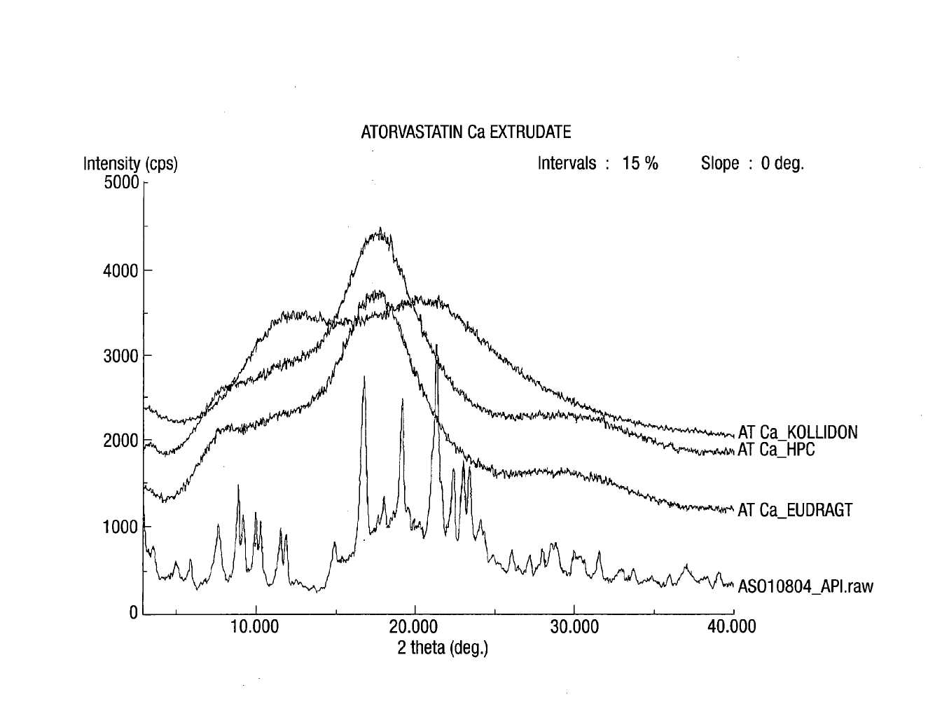

FIG. 1 indicates an X-ray powder diffractogram (XRD) of crystalline atorvastatin calcium and X-ray powder diffractograms (XRD) of amorphous atorvastatin calcium obtained as per the examples 1-3 of table 1 above.

### Example 9

A composition according to the invention comprised the following components:

| | | Qty/Tab |
|---|---|---|
| S. No | Ingredients | (mg) |
| | Dry mix | |
| 1.* | Ezetimibe | 10.0 |
| 2. | Mannitol IP | 494.63 |
| 3. | Low substituted Hydroxy propyl | 30.0 |
| | cellulose NF | |
| | Binder | |
| 4. | Hypromellose | 16.00 |
| | (HPMC 6cps) | |
| 5. | Purified water | — |
| | Extragranular | |
| 6.# | Atorvastatin calcium | 43.37 |
| 7. | Calcium carbonate | 10.00 |
| 8. | Low substituted Hydroxy propyl | 20.00 |
| | cellulose NF | |
| 9. | Talc | 8.00 |
| 10. | Calcium stearate | 8.00 |
| | Coating | |
| 11. | Opadry II 85G53348 Orange | 12.0 |
| 12. | Purified Water IP | q.s. |
| | TOTAL | 652 |

1. Ezetimibe, hydroxylpropyl cellulose (HPC low substituted), mannitol were mixed and granulated with HPMC solution.
2. The granules obtained in (1) were dried, sized and blended with Atorvastatin calcium, HPC low substituted, calcium carbonate, talc and calcium stearate.
3. The above blend was compressed into tablets and finally coated.

### Example 10

The formulation from example 10 was subjected to the accelerated and long term stability studies with the result confirming that the formulation is stable with regards to the following quality parameters:

| | | Long term | Accelerated |
|---|---|---|---|
| | | condition after | condition after |
| Condition | | period of 12 weeks | period of 12 weeks |
| Assay | Atorvastatin | 99.53% | 100.53% |
| | Ezetimibe | 98.98% | 98.59% |
| Dissolution | Atorvastatin | 95.57% at 60 min | 96.57% at 60 min |
| | Ezetimibe | 98.07% at 60 min | 95.95% at 60 min |

### Example 11

A composition according to the invention comprised the following components:

| | QUANTITY |
|---|---|
| INGREDIENTS | (mg/tab) |
| Drug layer: | |
| Fenofibrate | 160.00 |
| Atorvastatin calcium | 80.00 |
| Silicon dioxide | 5.00 |
| Polymer layer: | |
| Kollidon VA64 | 720.00 |
| (PVP: vinyl acetate) | |
| Sorbitan monolaurate | 15.00 |
| Blending: | |
| Calcium carbonate | 20.00 |
| Lactose | 160.00 |
| Microcrystalline | 100.00 |
| cellulose(Avicel 102) | |
| Crospovidone | 100.00 |
| Talc | 10.00 |
| Silicon dioxide | 10.00 |
| Lubrication: | |
| Sodium stearyl fumarate | 20.00 |
| Film coating | |
| Ready colour mix system | 20.00 |
| Purified Water IP | q.s. |
| Total | 1420.00 |

1. Atorvastatin calcium and fenofibrate with small amounts of silicon dioxide were sieved, sifted and mixed together in a mixer.
2. Kollidon VA 64 (6:4) was mixed separately with span 20 in a granulator and the mixture was then sifted.
3. The contents obtained above in (1) and (2) were mixed and was subjected to hot melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of calcium carbonate, lactose, crospovidone and microcrystalline cellulose and further lubricated with sodium stearyl fumarate.
4. The granules obtained were compressed to form a tablet which was film coated.

### Example 12

A composition according to the invention comprised the following components:

| | |
|---|---|
| | QUANTITY |
| INGREDIENTS | (mg/tab) |

ATORVASTATIN LAYER

| | |
|---|---|
| Drug Premix | |
| Atorvastatin calcium | 80.00 |
| Silicon dioxide | 4.00 |
| Polymer premix | |
| Kollidon VA-64 | 320.00 |
| (PVP: vinyl acetate) | |
| Sodium monolaurate | 16.00 |
| Blending | |
| Calcium carbonate | 20.00 |
| Mannitol | 160.00 |
| Low substituted | 80.00 |
| hydroxypropyl cellulose | |
| Lubrication | |
| Calcium stearate | 20.00 |

FENOFIBRATE LAYER

| | |
|---|---|
| Drug premix | |
| Fenofibrate | 160.00 |
| Starch | 125.00 |
| Kollidon CLM | 110.00 |
| Blending | |
| Sodium Lauryl Sulphate | 10.00 |
| Yellow oxide of iron | 1.00 |
| Kollidon CLM | 49.00 |
| Lactose | 180.00 |
| Talc | 10.00 |
| Binder | |
| Starch | 35.00 |
| Poly vinyl pyrrolidone K-30 | 10.00 |
| Lubrication | |
| Sodium stearyl fumarate | 10.00 |
| Film coating | |
| Ready colour mix system | 20.00 |
| Purified Water IP | qs |
| Total | 1420.00 |

1. Atorvastatin calcium with small amount of colloidal silicon dioxide was sieved, sifted and mixed together in a mixer.
2. Kollidon VA 64 was mixed separately with Sodium mono laurate in a granulator and the mixture was then sifted.
3. The contents obtained above in (1) and (2) were mixed and was subjected to hot melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of calcium carbonate, mannitol, low substituted hydroxylpropyl cellulose and further lubricated with calcium stearate.
4. Fenofibrate was mixed with pre-sieved and pre-sifted amounts of lactose, starch, Kollidon CLM, sodium lauryl sulphate, yellow oxide of iron, talc. Binder Starch and polyvinylpyrrolidone K-30 were added to the drug premix and granulated. The granules were lubricated with sodium stearyl fumarate.
5. The granules obtained in (3) and (4) were compressed together to form a bilayer tablet which was then film coated.

### Example 13

A composition according to the invention comprised the following components:

| | |
|---|---|
| | QUANTITY |
| INGREDIENTS | (mg/tab) |

ATORVASTATIN GRANULATE

| | |
|---|---|
| Drug premix | |
| Atorvastatin calcium | 80.00 |
| Silicon dioxide | 24.00 |
| Polymer mix | |
| Kollidon VA-64 | 320.00 |
| (PVP: vinyl acetate) | |
| Sodium monolaurate | 16.00 |

FENOFIBRATE GRANULATE

| | |
|---|---|
| Drug premix | |
| Fenofibrate | 160.00 |
| Lactose | 180.00 |
| Starch | 125.00 |
| Blending | |
| Kollidon CLM | 60.00 |
| Sodium lauryl sulphate(SLS) | 10.00 |
| Binder | |
| Starch | 35.00 |
| Polivinyl pyrrolidone K-30 | 10.00 |

EXTRAGRAULAR LAYER

| | |
|---|---|
| Kollidon CLM | 30.00 |
| Calcium carbonate | 20.00 |
| Talc | 10.00 |
| Sodium stearyl fumarate | 10.00 |
| Lactose | 110.00 |
| Film coating | |
| Ready colour mix system | 20.0 |
| Purified Water IP | qs |
| Total | 1220.00 |

1. Atorvastatin calcium and silicon dioxide were sifted & mixed together in a mixer.
2. Kollidon VA 64 was mixed separately with Sodium mono laurate in a granulator and the mixture was then sifted.
3. The contents obtained in (1) and (2) were mixed and subjected to hot melt extrusion (HME) wherein the melting temperature for the extrusion process ranges from 70 to 200° C., with the molten mass thus obtained was collected on a conveyor where it was cooled to form extrudates and these extrudates on further milling were converted in the form of granules.
4. Fenofibrate granules were prepared by mixing lactose, starch, kollidon CLM, sodium lauryl sulphate, polyvinyl pyrrolidone.
5. Amorphous Atorvastatin granules and fenofibrate granules were mixed with extragranular components such as kollidon CLM, calcium carbonate, talc, sodium stearyl fumarate, lactose were mixed and compressed together to form a tablet which was then film coated.

### Example 14

A composition according to the invention comprised the following components:

| | |
|---|---|
| | QUANTITY |
| INGREDIENTS | (mg/tab) |

Fenofibrate Granulate

| | |
|---|---|
| Fenofibrate | 160.00 |
| Kollidon VA-64 | 480.00 |
| (PVP: vinyl acetate) | |
| Silicon dioxide | 5.00 |

Atorvastatin Granulate

| | |
|---|---|
| Drug premix | |
| Atorvastatin calcium | 80.00 |
| Silicon dioxide | 5.00 |
| Polymer premix | |
| Kollidon VA-64 | 320.00 |
| (PVP: vinyl acetate) | |
| Sodium mono laurate | 16.00 |
| Blending | |
| Mannitol | 200.00 |
| Calcium carbonate | 20.00 |
| Low substituted hydroxyl | 80.00 |
| propyl cellulose LH11 | |
| Talc | 4.00 |
| Lubrication | |
| Calcium stearate | 30.00 |
| Film coating | |
| Opadry II | 20.0 |
| Purified Water IP | Qs |
| Total | 1420.00 |

1. Fenofibrate was mixed with silicon dioxide and Kollidon VA 64 and the extrudates were prepared by hot melt extrusion method (HME). These extrudates on further milling were converted in the form of granules.
2. Atorvastatin calcium with small amount of silicon dioxide was sieved, sifted and mixed together in a mixer.
3. Kollidon VA 64 was mixed separately with Sodium mono laurate in a granulator and the mixture was then sifted.
4. The contents obtained above in (2) and (3) were mixed and was subjected to hot melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of calcium carbonate, mannitol, low substituted hydroxylpropyl cellulose, talc and further lubricated with calcium stearate.
5. The granules obtained in (1) and (4) were mixed and compressed together to form tablet which was then film coated.

### Example 15

| | |
|---|---|
| | QUANTITY |
| INGREDIENTS | (mg/tab) |

EZETIMIBE LAYER

| | |
|---|---|
| Dry mix | |
| Ezetimibe | 10.00 |
| Mannitol 25 | 118.00 |
| Hydroxypropyl cellulose (Low substituted) | 5.00 |
| Yellow iron oxide | |
| Binder | |
| HPMC 6 cps | 4.00 |
| Purified water | q.s. |
| Lubrication | |
| Prosolve SMCC 90 | 50.00 |
| Microcrystalline cellulose | 109.8 |
| Calcium stearate | 3.00 |
| Total Weight | 300.00 |

ATORVASTATIN LAYER

| | |
|---|---|
| Drug premix | |
| Atorvastatin calcium | 43.37 |
| Kollidon VA 64 | 160.00 |
| Aerosil 200 | 2.50 |
| Span 20 | 8.00 |
| Blending & Lubrication | |
| Calcium carbonate | 25.00 |
| Ac-Di-Sol | 90.00 |
| Hydroxypropyl cellulose (Low substituted) | 100.00 |
| Aerosil 20 | 6.00 |
| Prosolve SMCC 90 | 155.13 |
| Calcium stearate | 10.00 |
| Total Weight | 600.00 |

### Procedure:

1. Ezetimibe, mannitol and small amount of Hydroxypropyl cellulose (Low substituted) and yellow iron oxide were sieved, sifted and mixed together in a mixer.
2. A separate solution of HPMC 6 cps was prepared in sufficient purified water.
3. The contents obtained above in (1) and (2) were mixed and was subjected to hot melt extrusion (HME) wherein the melting temperature for the extrusion process ranges from 70 to 200° C., with the molten mass thus obtained was collected on a conveyor where it was cooled to form extrudates and these extrudates on further milling were converted into granules which was followed by addition of prosolve SMCC 90, microcrystalline cellulose and further lubricated with calcium stearate.
4. Atorvastatin calcium was mixed with pre-sieved and pre-sifted amounts of Kollidon VA 64, Aerosil 200 and span 20. Blending agents such as calcium carbonate, Ac-Di-sol, Hydroxypropyl cellulose (Low substituted), Aerosil 200 and prosolve SMCC 90 were added to the drug premix and granulated. The granules were lubricated with calcium stearate.
5. The granules obtained in (3) and (4) were compressed together to form a bilayer tablet which was then film coated.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a polymer” includes a single polymer as well as two or more different polymers, reference to a “plasticizer” refers to a single plasticizer or to combinations of two or more plasticizer, and the like.

It will be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention.

1. A pharmaceutical composition comprising one or more cholesterol reducing agents in an amorphous form, wherein at least one agent is converted in-situ to an amorphous form during the manufacture of the composition.

2. The pharmaceutical composition according to claim 1, further comprising a polymer.

3. A composition comprising an extruded article containing one or more cholesterol reducing agents in an amorphous form in combination with a polymeric carrier, and optionally at least one pharmaceutically acceptable excipient.

4. The composition according to claim 3, wherein at least one amorphous agent is formed in situ during the manufacture of the composition.

5. The composition according to claim 2, wherein the ratio of agent to polymer is from 1:1 to 1:6 on a weight basis.

6. The composition according to claim 2, comprising 2 to 80 wt % polymer.

7-8. (canceled)

9. A pharmaceutical composition obtainable from a composition according to claim 3, optionally in combination with at least one pharmaceutically acceptable excipient.

10. The composition according to claim 1, wherein the cholesterol reducing agent comprises at least one HMG-CoA inhibitor, at least one cholesterol absorption inhibitor and/or at least one fibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

11. The composition according to claim 1, wherein the cholesterol reducing additive comprises at least one HMG CoA reductase inhibitor and/or at least one fibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

12. The composition according to claim 10, wherein the fibrate is selected from benzafibrate, gemfibrozil, fenofibrate, simfibrate, ronifibrate, ciprofibrate, etofibrate, clofibrate, clinofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

13. The composition according to claim 10, wherein the fibrate is fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

14. The composition according to claim 1, wherein the cholesterol reducing agent comprises at least one HMG CoA reductase inhibitor and/or at least one cholesterol absorption inhibitor or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

15. The composition according to claim 10, wherein the cholesterol absorption inhibitor is ezetimibe or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

16. The composition according to claim 10, wherein the HMG CoA reductase inhibitor is selected from cerivastatin, atorvastatin, lovastatin, simvastatin, rosuvastatin, pravastatin, mevastatin, fluvastatin, rivastatin, pitavastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

17. The composition according to claim 10, wherein the HMG CoA reductase inhibitor is atorvastatin or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

18. The composition according to claim 1, wherein the cholesterol reducing agent comprises atorvastatin calcium and ezetimibe or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

19. The composition according to claim 1, wherein the cholesterol reducing active comprises atorvastatin calcium and fenofibrate or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

20. The composition according to claim 1, obtainable by melt extruding at least one cholesterol reducing agent along with at least one pharmaceutically acceptable polymer, and optionally one or more other pharmaceutically acceptable excipients, to form an extrudate, and optionally admixing the extrudate with one or more further pharmaceutically acceptable excipients.

21-22. (canceled)

23. The composition according to claim 1, effective for use in treating dyslipidemia, hyperlipidemia, hypercholesterolemia, atherosclerosis, arteriosclerosis, cardiovascular disease, coronary artery disease, coronary heart disease, vascular disorder and/or a related disorder.

24. The method of making a pharmaceutical composition as defined in claim 1, containing one or more amorphous cholesterol reducing agents, comprising blending the agents with a pharmaceutically acceptable polymer, and hot melt extruding the blend to form an extrudate.

25. The method according to claim 24, wherein at least one cholesterol reducing agent is blended with the polymer, and the amorphous form of said active is formed in-situ during the hot melt extrusion.

26-35. (canceled)

36. The method according to claim 24, comprising processing the extrudate and optionally mixing it with one or more additives to form the pharmaceutical composition.

37. The method according to claim 24, comprising cutting the extrudate to a desired shape to form the pharmaceutical composition.

38. The method according to claim 24, comprising treating the extrudate to form a granulate.

39. The method according to claim 38, comprising filling the granulate into a capsule, or compressing the granulate to form a tablet.

40. (canceled)
